(19) United States
(12) Patent Application Publication
(10) Pub. No.: US 2022/0087877 A1
(43) Pub. Date: Mar. 24, 2022

(54) NONWOVEN WEB MATERIAL HAVING BONDING FAVORABLE FOR MAKING DIRECTIONAL STRETCH LAMINATE, AND DIRECTIONAL STRETCH LAMINATE

(71) Applicant: The Procter & Gamble Company, Cincinnati, OH (US)

(72) Inventors: Fred Naval Desai, Fairfield, OH (US); Olaf Erik Alexander Isele, West Chester, OH (US); Ryo Minoguchi, Cincinnati, OH (US); Jill Trygier, West Chester, OH (US)

(21) Appl. No.: 17/539,291

(22) Filed: Dec. 1, 2021

Related U.S. Application Data
(63) Continuation of application No. 15/586,358, filed on May 4, 2017.
(60) Provisional application No. 62/331,650, filed on May 4, 2016.

(51) Int. Cl. A61F 13/49; B32B 5/02; B32B 15/14; B32B 15/10; B32B 5/26; B32B 21/08; B32B 7/12; B32B 3/10; B32B 27/12; D04H 1/559; B32B 25/10; B32B 15/12; B32B 27/08; B32B 21/06; B32B 3/28; B32B 27/10; B32B 7/14; D04H 3/04; B32B 7/08; B32B 27/32; B32B 21/10; B32B 5/24; B32B 5/12; B32B 15/08; B32B 29/00; B32B 15/04; B32B 21/04; B32B 3/30; B32B 29/02; D04H 1/4382; D04H 1/42; D04H 1/4374; D04H 1/54; D04H 1/74

(52) U.S. Cl. CPC A61F 13/49012; D04H 1/43828; B32B 15/14; B32B 15/10; B32B 5/26; B32B 21/08; B32B 7/12; B32B 3/10; A61F 13/49011; B32B 27/12; D04H 1/559; B32B 25/10; B32B 15/12; A61F 13/4902; B32B 27/08; B32B 21/06; B32B 3/28; B32B 27/10; B32B 7/14; D04H 3/04; B32B 7/08; B32B 27/32; B32B 21/10; B32B 5/24; B32B 5/12; B32B 15/08; B32B 29/005; B32B 5/024; B32B 15/043; B32B 21/042; B32B 3/30; B32B 29/02; D04H 1/43832; D04H 1/42; D04H 1/4374; D04H 1/5405; D04H 1/74; B32B 2307/306; B32B 2270/00; B32B 2262/02; B32B 2307/51; B32B 2262/0276; B32B 2307/734; B32B 2262/0253; B32B 2255/10; B32B 2307/54; B32B 2307/546; B32B 2262/0261; B32B 2597/00; B32B 2439/70; B32B 2255/205; B32B 2555/02; B32B 2307/714; B32B 5/022

(57) ABSTRACT

A stretch laminate is disclosed. The stretch laminate may include a first layer; a second layer; and one or more elastic members disposed between the first layer and the second layer. The first layer may include a nonwoven web material bearing a pattern of thermal bonds, including a plurality of bonds each having a length and a width, the length being oriented perpendicularly to the stretch direction and being greater than the width, for a majority of the bonds. The elastic member(s) may be joined with the first layer and the second layer while pre-strained in the stretch direction; when the stretch laminate is in a relaxed condition, at least the first layer may include a plurality of gathers comprising elongate ridges and valleys oriented transversely to the stretch direction. A disposable absorbent pant having an elasticized belt structure including the stretch laminate, is also disclosed.

### CROSS REFERENCE TO RELATED APPLICATIONS

This application is a continuation of, and claims priority under 35 U.S.C. § 120 to, U.S. patent application Ser. No. 15/586,358, filed on May 4, 2017, which claims the benefit, under 35 USC § 119(e), of U.S. Provisional Patent Application Ser. No. 62/331,650, filed on May 4, 2016, the entire disclosures of which are fully incorporated by reference herein.

### BACKGROUND OF THE INVENTION

In recent years populations in many developed countries have shifted toward middle-aged and older demographic groups. These demographic groups represent markets with relatively increased demands for products and services addressed to concerns associated with aging.

One such concern is adult urinary incontinence. Urinary incontinence can result from or be exacerbated by a variety of health conditions, or even normal experiences such as childbearing.

Disposable absorbent pants for persons suffering from urinary incontinence have been marketed for a number of years. These products have traditionally been very similar to disposable baby diapers or disposable children's training pants, the main difference being size. One design type is known as the “belted” or “balloon” type pant, which is formed of a broad belt that encircles the wearer's waist and lower torso, bridged by a structure that connects front and rear belt portions through the wearer's crotch area. The crotch structure includes an absorbent structure designed to receive, contain and retain urine until the time the pant is changed. The belt is typically formed of a stretch laminate material.

Due to their design and method of manufacture, the products may visually resemble a disposable baby diaper or training pant, rather than an ordinary undergarment. The crotch structure may tend to be bulky as a result of the presence of absorbent materials. The structure may have the appearance of a mass-produced disposable article, like a disposable child diaper. The belt structure, typically formed of a stretch laminate material, may also have a bulky, mass-produced, diaper-like appearance.

This unfortunate resemblance has been a source of anxiety and discomfort for users. The bulk may cause outer clothing to fit poorly, or make it visibly obvious that an absorbent undergarment is being worn. Many users may be unhappy using products that may be associated with aging and loss of control of bodily functions.

In these circumstances, any improvement to traditional designs and materials for adult incontinence pants, that is efficient for manufacturing while providing an appearance and feel more closely resembling those of an ordinary undergarment, may provide competitive advantages to the manufacturer thereof.

### BRIEF DESCRIPTION OF THE DRAWINGS

FIG. 1 is a perspective view of one example of a balloon pant.

FIG. 2 is a perspective view of another example of a balloon pant.

FIG. 3 is a schematic plan view of a balloon pant precursor structure, prior to joining of the front and rear sections of the belt.

FIG. 4 is a schematic, exploded perspective view of components of a belt.

FIG. 5A is a plan view depiction of an example of a bond pattern.

FIG. 5B is a plan view depiction of another example of a bond pattern.

FIG. 5C is a plan view depiction of another example of a bond pattern.

FIGS. 5D-5I are enlarged plan view depictions of additional examples of bond patterns.

FIG. 6 is an enlarged view of a portion of the bond pattern depicted in FIG. 5A.

FIG. 7 is a plan view of a portion of a stretch laminate bearing an example of a bond pattern, the laminate depicted in a stretched condition.

FIG. 8 is a plan view of the portion of stretch laminate depicted in FIG. 7, following elastic contraction of elastic members along a stretch direction, and formation of ruffles or gathers.

FIG. 9 is a cross section of a portion of the stretch laminate depicted in FIG. 8, taken along line 9-9 in FIG. 8.

FIG. 10 is a cross section of a portion of the stretch laminate depicted in FIG. 8, taken along line 10-10 in FIG. 8.

### DETAILED DESCRIPTION OF EXAMPLES

### Definitions

“Cross direction” (CD)—with respect to the making of a nonwoven web material, the nonwoven material itself, a laminate thereof, or an article in which the material is a component, refers to the direction along the material substantially perpendicular to the direction of forward travel of the material through the manufacturing line in which the material and/or article is manufactured.

Throughout the present description, a material or composite of materials is considered to be “elastic” or “elastomeric” if, when a biasing force is applied to the material, the material or composite can be extended to an elongated length of at least 150% of its original relaxed length (i.e. can extend at least 50%), without rupture or breakage which substantially damages the material or composite, and when the force is removed from the material or composite, the material or composite recovers at least 40% of such elongation. In various examples, when the force is removed from an elastically extensible material, the material or composite may recover at least 60% or even at least 80% of its elongation.

The “stretch direction” of a stretch laminate is the direction along which the laminate will most readily undergo elastic stretch and contraction. In a stretch laminate in which one or more elastic members are incorporated into the laminate while in a pre-strained condition, the stretch direction is the direction along which the elastic member(s) are pre-strained. The “trans-stretch direction” of a stretch laminate is the direction perpendicular to the stretch direction.

“Film” means a skin-like or membrane-like layer of material formed of one or more polymers, which does not have a form consisting predominately of a web-like structure of consolidated polymer fibers and/or other fibers.

“Lateral”—with respect to a pant and its wearer, refers to the direction generally perpendicular with the wearer's standing height, or the horizontal direction when the wearer is standing.

For purposes herein, the length (L) and width (W) of a bond shape are the trans-stretch direction and stretch direction (SD) dimensions, respectively, of a rectangle having the least possible area while entirely circumscribing the bond shape, and having two of its sides aligned parallel with the stretch direction SD.

“Longitudinal”—with respect to a pant and its wearer, refers to the direction generally parallel with the wearer's standing height, or the vertical direction when the wearer is standing. “Longitudinal” is also the direction generally parallel to a line extending from the midpoint of the front waist edge to the midpoint of the rear waist edge.

“Machine direction” (MD)—with respect to the making of a nonwoven web material, the nonwoven material itself, or a laminate thereof, refers to the direction along the material or laminate substantially parallel to the direction of forward travel of the material or laminate through the manufacturing line in which the material or laminate is manufactured.

“Machine direction bias,” with respect to the fibers forming a nonwoven web, means that a majority of the fibers, as situated in the web and unstretched, have lengths with machine direction vector components that are greater than their cross direction vector components.

A “nonwoven” is a manufactured sheet or web of directionally or randomly oriented fibers which are first formed into a batt and then consolidated and bonded together by friction, cohesion, adhesion or one or more patterns of bonds and bond impressions created through localized compression and/or application of pressure, heat, ultrasonic or heating energy, or a combination thereof. The term does not include fabrics which are woven, knitted, or stitch-bonded with yarns or filaments. The fibers may be of natural and/or man-made origin and may be staple and/or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes including but not limited to meltblowing, spunbonding, spunmelting, solvent spinning, electrospinning, carding, film fibrillation, melt-film fibrillation, airlaying, dry-laying, wetlaying with staple fibers, and combinations of these processes as known in the art. The basis weight of nonwoven fabrics is usually expressed in grams per square meter (gsm).

“z-direction,” with respect to a web, means generally orthogonal or perpendicular to the plane approximated by the web along the machine and cross direction dimensions.

Although examples of the structure of the invention are described herein as used to form the belt of a belt- or balloon-type absorbent pant, it will be appreciated that examples may be used to form other components of pants, diapers, other wearable articles, and other products as well.

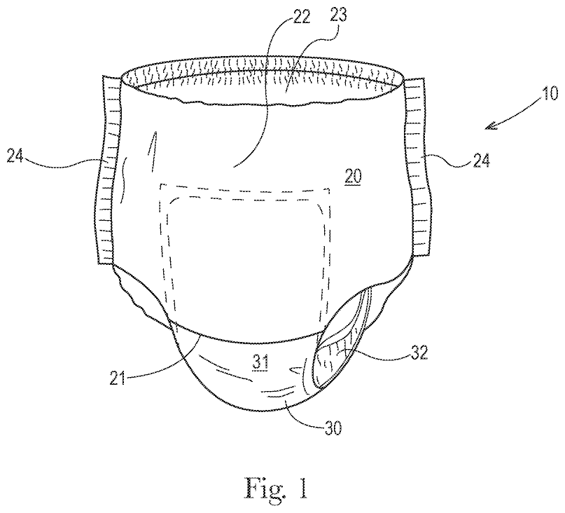

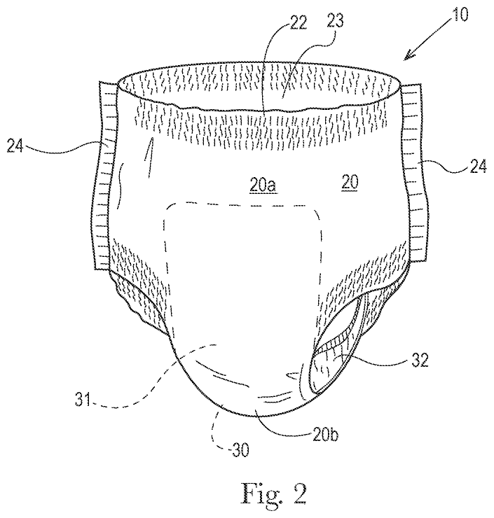

FIGS. 1 and 2 depict examples of belt- or balloon-type absorbent pants 10. The structure may include a belt portion 20 and a central chassis 30. Central chassis 30 may include any combination of components found in disposable diapers and absorbent pants, including but not limited to a liquid impermeable backsheet 31, a liquid permeable topsheet (not shown), an absorbent core structure (not shown), and elasticized barrier cuffs 32. Examples and descriptions of components and configurations of a central chassis may be found in U.S. patent application Ser. No. 13/764,945, wherein the chassis described includes components and features that may be included in central chassis 30.

In the example shown in FIG. 1, belt portion 20 stops short of the crotch region 12 of the pant, at lower edge 21; this configuration is sometimes called a “multipiece” configuration. In the example shown in FIG. 2, belt portion 20 is part of an outer structure that includes a belt portion 20a encircling the wearer's waist, an outer wrap portion 20b that overlies the central chassis to the outside thereof and wraps thereabout through the crotch region; this configuration is sometimes called a “unibody” configuration. The outer wrap portion 20b may be formed of a layer of nonwoven web, which also may serve as the outer layer of the belt portion 20a. The belt portion 20 may have front and rear portions 22, 23, which are joined together at seams 24.

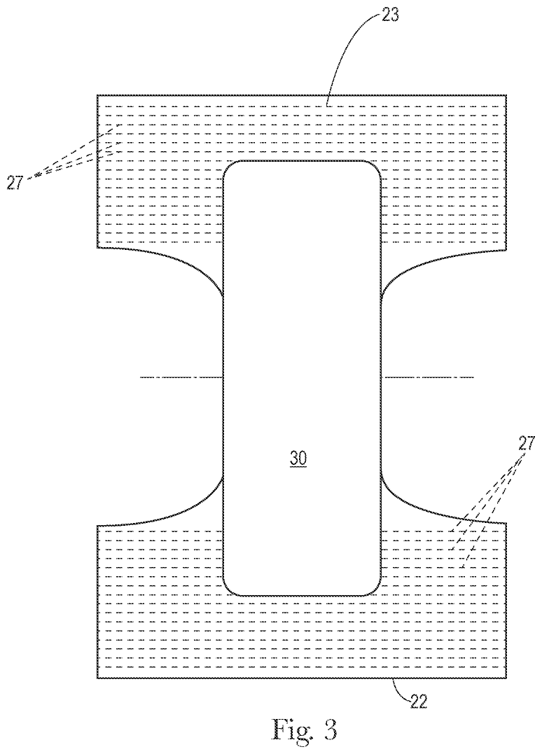

FIG. 3 schematically depicts a structure that is the precursor to a pant such as depicted in FIG. 2, prior to joining of front and rear portions 22, 23 at seams 24 as depicted in FIGS. 1A and 1B. Central chassis 30 overlies front and rear portions 22, 23 to the inside thereof.

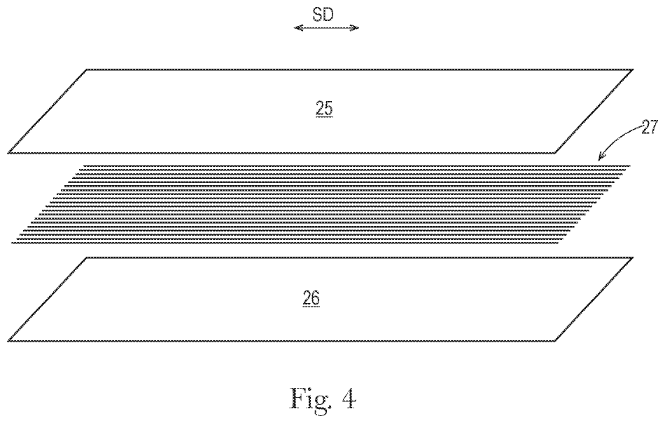

Referring to FIG. 4, a belt portion 20 may be formed of layers of nonwoven web 25, 26, which respectively form inner and outer layers of the belt. Suitable nonwoven web materials that may be useful in the present invention also include, but are not limited to, spunbond, spunlaid, meltblown, spunmelt, solvent-spun, electrospun, carded, film fibrillated, melt-film fibrillated, air-laid, dry-laid, wet-laid staple fibers, and other and other nonwoven web materials formed in part or in whole of polymer fibers, as known in the art. The nonwoven web may be formed predominately of polymeric fibers. In some examples, suitable non-woven fiber materials may include, but are not limited to polymeric materials such as polyolefins, polyesters, polyamide, or specifically, polypropylene (PP), polyethylene (PE), poly-lactic acid (PLA), polyethylene terephthalate (PET) and/or blends thereof. In some examples, the fibers may be formed of PP/PE blends such as described in U.S. Pat. No. 5,266,392, the disclosure of which is incorporated by reference herein. Nonwoven fibers may be formed of, or may include as additives or modifiers, components such as aliphatic polyesters, thermoplastic polysaccharides, or other biopolymers. Further useful nonwovens, fiber compositions, formations of fibers and nonwovens and related methods are described in U.S. Pat. Nos. 6,645,569; 6,863,933 and 7,112,621; and in co-pending U.S. patent application Ser. Nos. 10/338,603; 10/338,610; and Ser. No. 13/005,237, the disclosures of which are incorporated by reference herein.

The individual fibers may be monocomponent or multicomponent. The multicomponent fibers may be bicomponent, such as in a core-and-sheath or side-by-side arrangement. Often, the individual components comprise polyolefins such as polypropylene or polyethylene, or their copolymers, polyesters, thermoplastic polysaccharides or other biopolymers.

According to one example, the nonwoven may comprise a material that provides good recovery when external pressure is applied and removed. Further, according to one example, the nonwoven may comprise a blend of different fibers selected, for example from the types of polymeric fibers described above. In some embodiments, at least a portion of the fibers may exhibit a spiral curl which has a helical shape. According to one example, the fibers may include bicomponent fibers, which are individual fibers each comprising different materials, usually a first and a second polymeric material. It is believed that the use of side-by-side bi-component fibers is beneficial for imparting a spiral curl to the fibers.

In order to enhance softness perceptions of the laminate, nonwovens may be treated by hydrojet impingement, which may also be known as hydroenhancement, hydroentanglement or hydroengorgement. Such nonwovens and processes are described in, for example, U.S. Pat. Nos. 6,632,385 and 6,803,103, and U.S. Pat. App. Pub. No. 2006/0057921, the disclosures of which are incorporated herein by reference.

Other examples of nonwoven web that may be useful in the present laminate may be an SMS web (spunbond-meltblown-spunbond web) made by Avgol Nonwovens LTD, Tel Aviv, Israel, under the designation XL-S70-26; an SSS (spunbond-spunbond-spunbond) web made by Pegas Nonwovens AS in Znojmo, Czech Republic, under the designation 18 XX 01 00 01 00 (where XX=the variable basis weight); an SSS web made by Gulsan Sentetik Dok San VE TIC AS, in Gaziantep, Turkey, under the designation SBXXFOYYY (where XX=the variable basis weight, and YYY=the variable cross direction width); an HESB (hydroenhanced spunbond) web made by First Quality Nonwovens Inc., in Hazelton, Pa., under the designation SEH2503XXX (where XXX=the variable cross direction width); and a bicomponent SS web.

A nonwoven web useful as a component to form one or both of layers 25, 26 may be bonded in a pattern of bonds. A batt of loose, e.g., spunlaid, fibers may be passed through the nip between a pair of calender bonding rollers and thereby consolidated and bonded in a pattern of bonds, to add tensile strength and dimensional stability, converting the batt of loose fibers to a coherent and useable nonwoven web material. The bonding may include a pattern of thermal bonds, mechanical bonds or adhesive bonds or a combination thereof, although in some circumstances thermal bonding may be preferred. Thermal bonds may be formed by supplying one or both of the calender rollers or accompanying equipment with a source of heating energy that functions to heat the fibers and cause them to melt and fuse beneath bonding projections in the nip between the calender bonding rollers. One or both of the rollers may be machined, etched or otherwise formed to have a pattern of shaped bonding projections extending radially outward from the cylindrical surface of the roller. When the rollers are maintained in suitably close proximity with their axes in parallel, the batt of fibers passing therebetween will be subjected to pressure concentrated in the nip beneath the bonding projections, and fibers passing through the nip and beneath the bonding projections will be deformed and at least partially fused (by application of heating energy), to form bonds. Each bond will have a shape, and the bonds will have a pattern and spacing, substantially corresponding to the shape, pattern and spacing of the bonding projections on the calender bonding roller.

Referring to FIG. 4, layers of nonwoven web 25, 26 may sandwich one or more elastic members such as a plurality of strands 27 of an elastomeric material, such as an elastane (for example, LYCRA HYFIT fiber, a product of Invista, Wichita, Kans.). Layers of nonwoven web 25, 26 may be joined together about elastic strands 27 by adhesive deposited between the layers, by thermal bonds, by compression bonds, or by a combination thereof. In other examples, the one or more elastic members may be strips or a section of film formed of elastomeric material.

The elastic members can also be formed from various other materials, such as but not limited to, rubbers, styrene ethylbutylene styrene, styrene ethylene propylene styrene, styrene ethylene ethylene propylene styrene, styrene butadiene styrene, styrene isoprene styrene, polyolefin elastomers, elastomeric polyurethanes, and other elastomeric materials known in the art, and combinations thereof. In some embodiments, the elastic members can be extruded strand elastics with any number of strands (or filaments). Elastic strands, if used, may be selected to have a decitex ranging from 50 to 2000, or any integer value for any decitex value in this range, or any range formed by any of these integer values. The elastic members may be in a form of film. Examples of films have been described extensively in prior patent applications (see, for example, U.S. Pat. App. Pub. No. 2010/0040826). The film may be created with a variety of resins combined in at least one of several sublayers, the latter providing different benefits to the film. Elastic members may also be in the form of scrim, strips or sections of tape of elastomeric material with their longer dimensions oriented along the stretch direction.

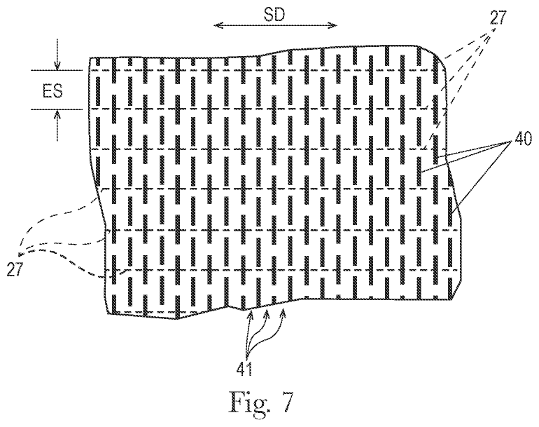

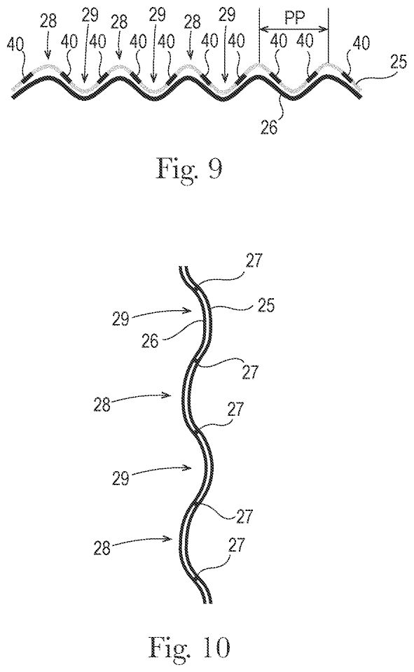

During manufacture of the belt structure, the one or more elastic members such as elastic strands 27, may be pre-strained lengthwise by a desired amount as they are being incorporated into the belt structure. Upon subsequent relaxation of the belt, the one or more elastic members, such as elastic strands 27, will contract toward their unstrained lengths. Referring to FIGS. 8-10, this causes the layers of nonwoven material 25, 26 to gather and form ruffles or gathers having ridges 28 (shown in FIG. 8 as light areas) and valleys 29 (shown in FIG. 8 as dark areas) generally transverse to the lengths of the elastic strands 27. The direction of this strain corresponds with the stretch direction of the laminate.

The size(s) and shape(s) of the ruffles or gathers will be affected, and may be manipulated, by design of the pattern of joined portions and/or bonding between the layers of nonwoven web 25, 26, with respect to each other and with respect to elastic strands 27.

In one example, a stretch laminate may incorporate elastic strands 27 as the elastic stretch mechanism. Elastic strands 27 may have adhesive applied to them prior to lamination (e.g., by a strand coating process), such that, when the web layers 25, 26 are brought together to sandwich the strands, the applied adhesive causes the web layers to be adhered about the strands to form the stretch laminate. The adhesive applied to the elastic strands may be the only adhesive used to hold the laminate together. Alternatively, or in addition, adhesive may be deposited upon one or both layers 25, 26 prior to lamination, and may be deposited in a pattern. Examples of methods for applying patterned deposits of adhesive to a nonwoven web substrate to enable manufacture of an elasticized laminate are described in U.S. Pat. No. 8,186,296. In one example, the adhesive pattern selected may be effected by design of a correspondingly designed roller. The pattern of adhesive to be applied may be designed to affect the size(s) and shape(s) of the ruffles or gathers. The layers 25, 26 may be adhesively joined and/or bonded to each other at the locations of adhesive deposits, and remain unjoined or unbonded, or free, of each other at other locations, such that they may move and shift slightly relative each other as the laminate is moved and stretched, as during wear of the article.

When bonding of one or both of layers 25, 26 is effected using thermal calender bonding, the joining and/or bonding pattern may be designed to affect the size(s) and shapes of the ruffles or gathers. It may be desired in some circumstances that a spunlaid nonwoven web be bonded with a pattern of thermal bonds to a bond area of from 5% to 20%. For purposes described herein it may be desired that bond area be from 8% to 15%. Patterned thermal bonding tends to enhance machine-direction and cross-direction strength and dimensional stability of the resulting bonded nonwoven web, which has benefits in downstream converting and processing operations, and adds tensile strength and robustness to a product in which the web is to form a component. However, thermal bonding also generally increases the stiffness of the resulting bonded nonwoven web. This may have adverse effects on the product consumer's perception of tactile softness of the product surfaces. For example, if the web is used as a layer of a belt structure of a pant product, stiffness imparted to the web may cause the consumer to negatively perceive the belt layer as stiff- or rough-feeling. For this reason, in some circumstances it may be desired to limit bond area to less than 16%, less than 12%, less than 10% or even less than 8%. It has been discovered, however, that imparting certain features as described herein to the bond pattern of a web to be used in a stretch laminate can mitigate the negative effects of stiffening the web, while providing advantages in addition to tensile strength.

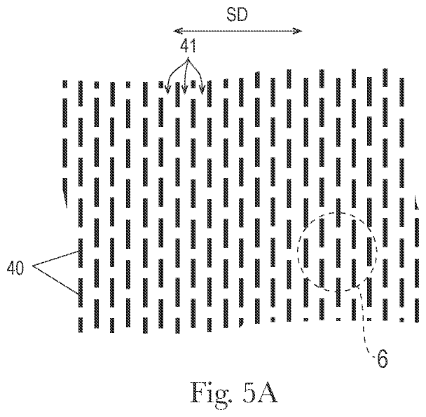

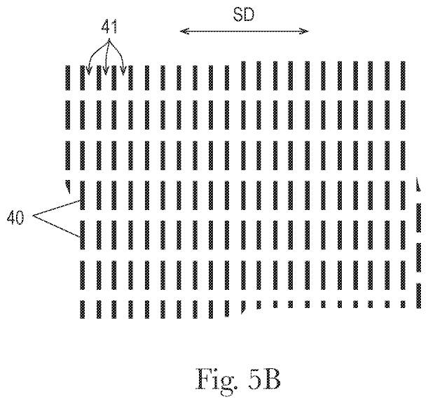

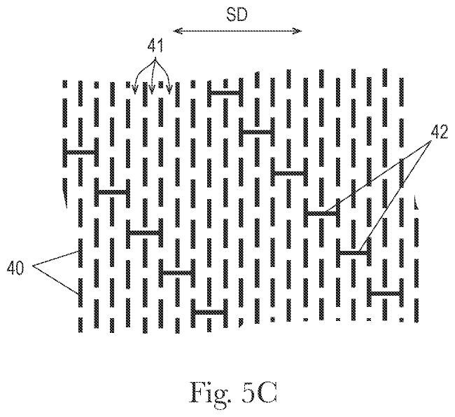

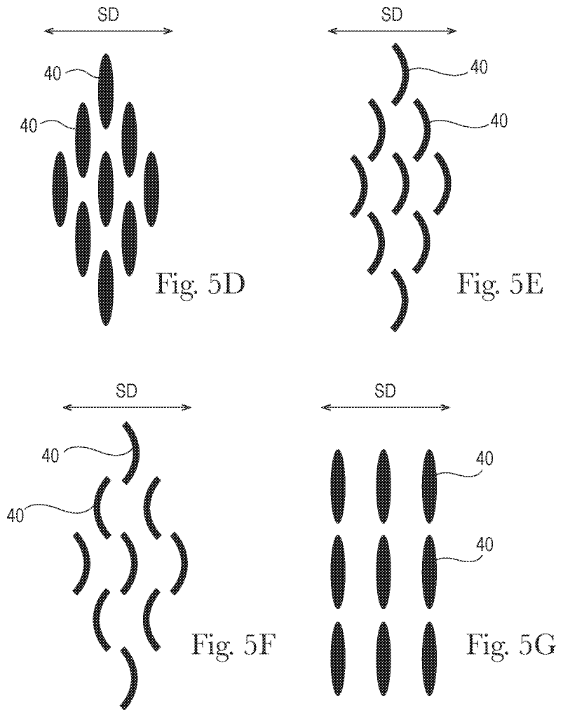

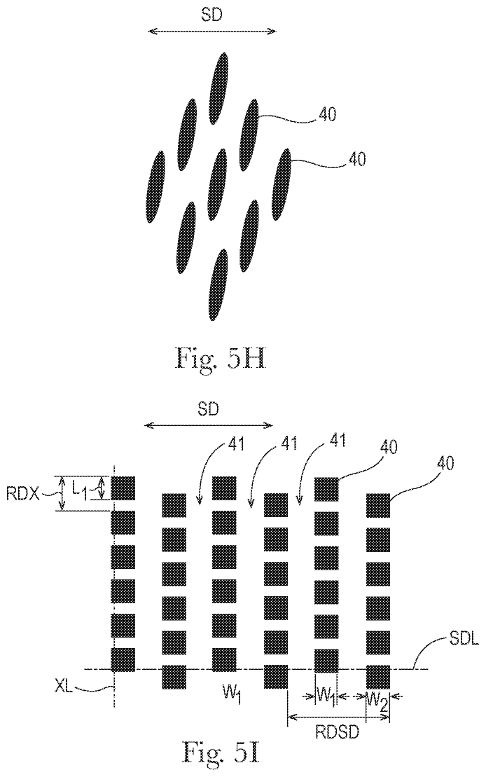

Referring to FIGS. 5A and 6, a thermal bonding pattern may include a plurality of individual bonds 40 having elongate shapes having a length dimension L measured along a trans-stretch direction (perpendicular to the stretch direction SD) and a width dimension W measured along a direction parallel with the stretch direction SD. The bond shapes and arrangement on the nonwoven will substantially correspond with the shapes and arrangement of the radially outwardmost surfaces of the bonding projections (sometimes called a “bonding pins”) on the calender roller used to create the bonds. The pattern may be either regular or irregular, along one or both of two perpendicular directions. Columns of individual bonds may be located at column repeat interval distances CI. Rows of individual bonds may be located at row repeat interval distances RI. Unbonded pathways 41 may be present between columns of bonds, and may extend indefinitely, or past only a plurality of rows, without interruption. Column angle α is the angle at the intersection between a line parallel with the stretch direction, and a line bordering an unbonded pathway 41 (tangent to the shapes in a column of bond shapes). Each unbonded pathway 41 has a width PW, measured along a direction parallel to the stretch direction. Width PW is the distance between two respective parallel lines bordering the unbonded pathway 41 (respectively tangent to the shapes in two adjacent columns of bond shapes), in which no portions of any bonds appear. FIGS. 5A and 6 are not to be deemed limiting or exclusive examples of suitable bonding patterns contemplated herein. Additional, alternative but non-limiting, examples are depicted in FIGS. 5B-5I.

It has been found that orienting stiffening columns of bonds such that they run substantially perpendicular to the stretch direction SD (such that unbonded pathways 41 also run substantially perpendicular to and for a substantial length along the stretch direction), beneficial effects upon formation of ruffles or gathers in layers 25 and/or 26 may be achieved. Within the region occupied by a bond 40 in a nonwoven web layer 25 and/or 26, the constituent fibers of the web have been melted, deformed, highly consolidated and fused together. As a result, within the bond shape the structure is stiffened relative the surrounding unbonded areas. The stiffened area will tend to be more resistive to bending and flexing than the surrounding unbonded areas. Conversely, the unbonded areas (comprising undeformed and unbonded fibers) will tend to more easily bend and flex relative the stiffened areas within the bonds. Thus, the web will more easily bend and flex along an unbonded pathway 41, while columns of bonds 40 will tend to remain relatively resistant to bending and flexing. These effects may be exploited via design of the bond pattern, to favorably affect formation of ruffles or gathers in the stretch laminate.

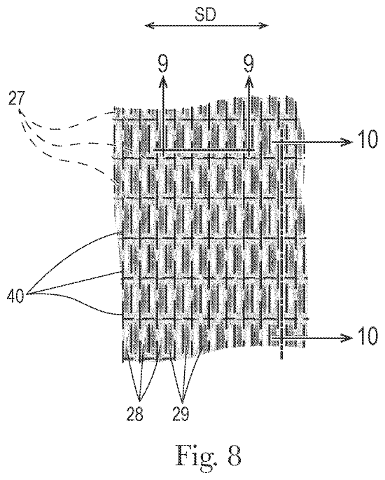

Referring to FIGS. 5 and 9, bonds 40 will tend to cause the web layer 25 to resist bending in the bonded areas, such that peaks and floors of ridges 28 and valleys 29 are more likely to form along the more flexible unbonded areas such as unbonded pathways 41. As noted above, substantial bonding (e.g., bonding area of greater than 4%) of nonwoven web layers of a stretch laminate may in some circumstances be undesirable because of perceived negative effects on stiffness and perceived softness of the material. However, when the bond pattern is configured as described herein, the stiffened (bonded) areas of the web may be effectively located away from the user's touch, by their locations on the sides of the ridges. The peaks of the ridges will be formed of unbonded, soft fibrous regions of the web, and protrude in the z-direction, imparting a soft tactile feel to the stretch laminate.

The shapes and dimensions of the bonds 40 may be configured for beneficial impact not only on tactile softness, but on formation and size of the ruffles or gathers. Currently marketed belt- or balloon-style pants that include longitudinally spaced elastic strands as the belt stretch mechanism tend to have a bulky, puffy appearance that may not be deemed desirable for an adult product. It has been found that selecting spacing of the elastic strands in conjunction with spacing and dimensions of the bonds 40 in a bonded nonwoven layer 25 and/or 26 can work to greatly reduce the size of the ruffles or gathers, and also enhance regularity and consistency of size and shape. This helps impart a neat, low-bulk, cloth-like appearance to the stretch laminate.

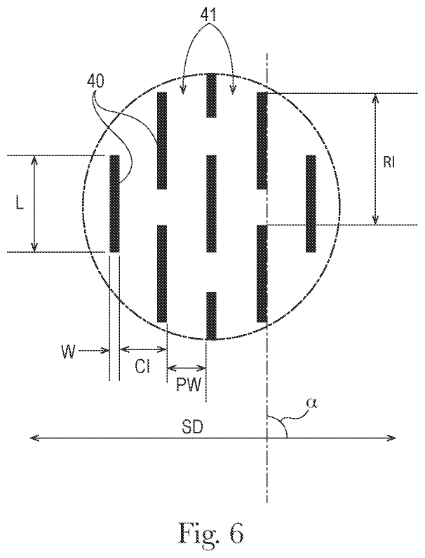

Referring to FIGS. 7-10, straight, parallel and spaced elastic strands 27 may be incorporated into a stretch laminate in a pre-strained condition, along a stretch direction SD. Referring to FIG. 8, upon completion of manufacture of the stretch laminate, and elastic contraction of elastic strands 27 toward their unstrained lengths (as indicated by the large arrows), they will draw layers 25 and 26 and cause them to contract along the stretch direction and form ruffles or gathers having ridges 28 and valleys 29. The dimensions of the bond shapes and bond pattern may be coordinated with the trans-stretch spacing S of the elastic strands 27 for various advantages.

Alone or in combination with any other features described herein, bond shape length L may be selected so as to be no less than 33% of row repeat interval RI, more preferably no less than 40%, 50%, 60% and even more preferably no less than 70% of row repeat interval RI. This feature contributes to orderly and regular formation of gathers with peaks and valleys extending along a direction perpendicular to the stretch direction. It may be desired that bond shape length L be no greater than 90% of row repeat interval RI. This ensures that the bonds are discrete (not indefinitely long), and avoids imparting too much stiffness to the web along a direction perpendicular to the stretch direction SD.

Alternatively, or in combination with any other features described herein, bond shape width W, may be selected to be from 5% to 50% of column repeat interval CI, more preferably from 8% to 40%, and still more preferably from 10% to 30% of column repeat interval CI. This feature strikes a balance between providing an appropriate width for bond-stiffened regions that will resist bending and thereby form the sides of ridges 28, while leaving an appropriate width for non-bonded, non-stiffened regions that will bend or flex to form the peaks and floors of ridges 28 and valleys 29 of gathers in the web material.

Alternatively, or in combination with any other features described herein, the pattern of bonds may be arranged such that the total of unbonded pathway widths PW for the pattern over a stretch direction distance over which the pattern repeats is from 50% to 95% of the stretch direction repeat length, more preferably from 60% to 92% of the stretch direction repeat length, and still more preferably from 70% to 90% of the stretch direction repeat length.

Referring to FIGS. 6 and 7, in combination with any other features described herein, bond row repeat interval RI may be related to trans-stretch elastic strand spacing ES such that RI is equal to or less than 2.0 times, more preferably 1.5 times, and even more preferably 1.0 times strand spacing ES. If RI is equal to or less than 1.0×ES, lines of web flex extending along the stretch direction SD will be present in at least the same number and frequency as the number of elastic strands 27 present, promoting suitable flexibility along the trans-stretch direction. In conjunction with this feature, bond length L may be equal or less than 2.0 times, more preferably equal to or less than 1.5 times, and even more preferably equal to or less than 1.0 times strand spacing ES.

For purposes of reducing the overall size of the ruffles or gathers formed, and in conjunction with any combination of the features described herein, it may be desired that the trans-stretch spacing ES of the elastic strands 27 be no greater than 14 mm, more preferably no greater than 10 mm, even more preferably no greater than 7 mm, and still more preferably no greater than 5 mm. (Herein, trans-stretch spacing of adjacent elastic strands is to be understood to refer to the distance between their longitudinal axes, not the distance between their nearest outer surfaces.) Through experimentation it has been determined that limiting spacing of elastic strands 27 in this way has the effect of promoting formation of ruffles or gathers that are relatively small, thereby promoting or enhancing a cloth-like appearance in the stretch laminate. This effect may be complimented and amplified by incorporating other features of a bond pattern in one or both of the nonwoven web layers 25, 26 as described herein.

In combination with any of the other features described herein, column repeat interval CI may be related to trans-stretch elastic strand spacing ES such the CI is no greater than 1.5 ES, more preferably no greater than 1.3 ES, 1.0 ES, 0.9 ES, and even more preferably no greater than 0.8 ES. This feature enables control of the size and regularity/uniformity of the ruffles or gathers formed relative the trans-stretch spacing of the elastic strands 27. If, for example, a stretch laminate has elastic strands spaced at ES=5.0 mm, this means that it may be desired that bond column repeat interval CI be 7.5 mm or less, 6.5 mm or less, 5.0 mm or less, 4.5 mm or less, or even 4.0 mm or less. This dimension, along with the extent of pre-strain imparted to the elastic strands 27 during formation of the stretch laminate, will further impact the stretch-direction size of the ruffles or gathers. For stretch laminates as contemplated herein, pre-strain in the elastic strands in the range from 50% to 200% is envisioned. (Herein, the pre-strain percentage amount is expressed as the calculation of the length of a section of the strand in the pre-strained condition minus the length of the same section of the strand in its relaxed condition (stretch distance), divided by length of the same section of the strand in its relaxed condition, times 100%.) For example, assuming a level of pre-strain in the elastic strands sufficient to cause the laminate to contract upon relaxation to approximately half of its stretch-direction, fully-stretched length (e.g., a pre-strain amount of approximately 100%), a bond column repeat interval CI will promote formation of ruffles or gathers upon relaxation of the stretch laminate that have a peak-to-peak dimension PP (see FIG. 9) of approximately half of bond column repeat interval CI. Under this circumstance, if bond column repeat interval CI is 7.5 mm or less, formation of regular, uniform ruffles or gathers having a peak-to-peak dimension of 3.8 mm or less, may be promoted. This is the stretch-direction relaxed width of the ruffles or gathers. From construction of prototypes and observation it has been determined that uniform and regular ruffles or gathers of such width or smaller visually appear quite small and thereby substantially contribute to imparting a cloth-like appearance to the stretch laminate.

In combination with any of the other features described herein, a majority of the bonds 40 in the pattern may have shapes with an aspect ratio of length to width equal to or greater than 1.0, more preferably equal to or greater than 1.5, even more preferably equal to or greater than 2.0, still more preferably equal or greater than 2.5, and most preferably equal to or greater than 3.5. This feature will contribute to promoting formation of regular and uniform ruffles or gathers with ridges and valleys oriented in the trans-stretch direction.

Another characteristic of a bond pattern that can provide a way to effect control over formation of ruffles or gathers for purposes herein may be described with reference to FIG. 5I. A bond pattern will have a stretch direction repeat interval RDSD measured along a line SDL parallel with the stretch direction SD. A bond pattern will have a trans-stretch direction repeat interval RDX measured along a line XL perpendicular to the stretch direction SD. Bonds 40 included within the stretch direction repeat interval RDSD may have, e.g., stretch direction widths W1, W2, W3, etc. Bonds 40 within the trans-stretch direction repeat interval RDX may have, e.g., trans-stretch direction lengths L1, L2, L3, etc. In a bond pattern suitable for purposes herein: a trans-stretch direction line XL may be identified, along which the ratio of the sum of the bond lengths (L1, L2, etc.) to the trans-stretch direction repeat interval (RDX) that includes the bond lengths, is greater than the ratio of the sum of the bond widths (W1, W2, etc.) to the stretch direction repeat interval (RDSD) that includes the bond widths, along any stretch direction line (SDL) along the pattern that can be identified. Expressed differently, in an example of a bond pattern suitable for purposes herein: a line XL perpendicular to the stretch direction can be identified, wherealong

(L1+L2+etc.)/RDX>(W1+W2+etc.)/RDSD,

for any line SDL that can be identified extending in the stretch direction. Expressed yet another way, a bond pattern in which a trans-stretch direction line along which bonds are arranged traverses a greater sum of bond lengths per unit line length along the web, than any sum of bond widths per unit line length crossed by any stretch direction line that can be identified. The effect of this configuration of bonds is substantially trans-stretch direction columnar stiffened regions alternating with substantially trans-stretch direction columnar flexible regions. This configuration tends to promote formation of ruffles or gathers with improved uniformity and regularity, with alternating ridges 28 and valleys 29 oriented along the flexible columnar regions, and sides or slopes along the stiffened columnar regions, as may be appreciated from FIG. 9. Assuming that the various examples of patterns depicted in FIGS. 5A-5I are proportionally accurate as reproduced from the drawings originally prepared for submission herewith, it can be appreciated that each satisfies this condition (or will, with minor adjustment to proportions), although these are only non-limiting, non-exclusive examples.

The columns of bond shapes may be configured to be substantially perpendicular to the stretch direction SD, e.g., in FIG. 6, column angle α is 90 degrees. It is not necessary that angle α is exactly 90 degrees, but it may be preferable that column angle α is from 70 to 110 degrees, more preferably from 80 to 100 degrees, even more preferably from 85 to 95 degrees, and most preferably from 87 to 93 degrees. This ensures orderly, regular formation of ruffles or gathers along a direction substantially perpendicular to the stretch direction.

A pattern that has unbonded pathways 41 that lie exactly along the cross-direction of the bonded nonwoven web material reflects a calender bonding roller with a corresponding arrangement of bonding projections. This arrangement pay increase the possibility for increased or undesired excess pressure beneath bonding projections that may result in non-uniform or defective bonds and/or roller and equipment wear, which may result from intermittent and rapid, step-wise changes in pressure in the nip resulting from intermittent absence and presence of bonding projections in the nip that may occur when trans-machine direction columns of bonding projections alternate with trans-machine direction unbonded pathways with no projections present on the cylindrical surface of the calender bonding roller.

Thus, when the machine direction of the bonded nonwoven web layer corresponds with the stretch direction, it may be desired that column angle α not be exactly 90 degrees. Alternatively or in addition, the calender bonding roller may include bonding projections that intermittently interrupt the unbonded pathways 41, resulting in formation of trans-stretch pathway interrupting bonds 42, as suggested in FIG. 5C. Interrupting bonds 42 reflect bonding projections that may be included to mitigate or eliminate the above-referenced rapid, step-wise changes in pressure in the nip between the calender bonding rollers.

The stretch direction SD of the stretch laminate may be parallel or perpendicular with the machine direction of manufacture of the nonwoven web layer components 25, 26. For nonwoven webs formed of continuously spun fibers, the fibers in the web often have a machine-direction bias as a result of the manner in which the fibers are spun and laid down on a moving belt. As a result of this machine-direction bias of the fibers, the nonwoven web may be imparted with anisotropic tensile strength properties, for example, machine-direction tensile strength is greater than cross-direction tensile strength. It has been determined, however, than when a bond pattern has a combinations of one or more features described above, the columns of bond shapes are substantially perpendicular to the machine direction, the rows of bond shapes are offset or staggered as suggested in FIG. 5A (in contrast to aligned as depicted in FIG. 5B), that anisotropy of tensile strength may be substantially reduced, and dimensional stability of the web may be substantially improved (particularly but without limitation, undesirable necking or neck-down behavior may be substantially reduced). As noted, tensile strength and dimensional stability of the nonwoven web are important for converting and processing operations, and for structural integrity and robustness of a product in which the nonwoven web is used as a component. Accordingly, it may be desired that the stretch direction of the stretch laminate be substantially parallel to (i.e., substantially the same as) the machine direction of the nonwoven web layer bearing a bond pattern as described herein.

In view of the description above, the following examples are contemplated:

1. A stretch laminate having a stretch direction and a trans-stretch direction perpendicular to the stretch direction, comprising:
a first web layer comprising a nonwoven web material bearing a pattern of thermal bonds comprising a plurality of bonds each having a length and a width, the length being oriented perpendicularly to the stretch direction and being greater than the width, for a majority of the bonds;
a second web layer; and
one or more elastic members disposed between the first web layer and the second web layer,

wherein the first web layer, the second web layer and the one or more elastic members together form the stretch laminate;

wherein the one or more elastic members have been joined with the first web layer and the second web layer while the one or more elastic members are pre-strained in the stretch direction; and

wherein, when the stretch laminate is in a relaxed condition, at least the first web layer comprises a plurality of gathers comprising elongate ridges and valleys oriented transversely to the stretch direction.

2. A stretch laminate having a stretch direction and a trans-stretch direction perpendicular to the stretch direction, comprising:
a first web layer comprising a nonwoven web material bearing a

pattern of thermal bonds comprising a plurality of bonds each having a length and a width, the length being oriented perpendicularly to the stretch direction;
a second web layer; and
one or more elastic members disposed between the first web layer and the second web layer,

wherein the first web layer, the second web layer and the one or more elastic members together form the stretch laminate;

wherein the one or more elastic members have been joined with the first web layer and the second web layer while the one or more elastic members are pre-strained in the stretch direction; and

wherein, the pattern of bonds has a stretch direction repeat interval RDSD measured along an identifiable first line SDL parallel with the stretch direction, a trans-stretch direction repeat interval RDX measured along a second line XL perpendicular to the stretch direction, individual ones of the bonds arranged along the second line XL and within the trans-stretch direction repeat interval RDX have lengths, individual ones of the bonds arranged along the first line SDL and within the stretch direction repeat interval RDSD have widths, and the ratio of the sum of the lengths to the trans-stretch direction repeat interval RDX is greater than the ratio of the sum of the widths to the stretch direction repeat interval RDSD, for any identifiable second line SDL; and

wherein, when the stretch laminate is in a relaxed condition, at least the first web layer comprises a plurality of gathers comprising elongate ridges and valleys oriented transversely to the stretch direction.

3. A stretch laminate having a stretch direction and a trans-stretch direction perpendicular to the stretch direction, comprising:
a first web layer comprising a nonwoven web material bearing a

pattern of thermal bonds comprising a plurality of bonds each having a length and a width, the length being oriented perpendicularly to the stretch direction;
a second web layer; and
one or more elastic members disposed between the first web layer and the second web layer,

wherein the first web layer, the second web layer and the one or more elastic members together form the stretch laminate;

wherein the one or more elastic members have been joined with the first web layer and the second web layer while the one or more elastic members are pre-strained in the stretch direction;

wherein, when the stretch laminate is in a relaxed condition, at least the first web layer comprises a plurality of gathers comprising elongate ridges and valleys oriented transversely to the stretch direction; and

wherein, the majority of the bonds in the pattern of bonds have an aspect ratio of length to width of at least 2.

4. The stretch laminate of any of examples 1-3 wherein each ridge of the plurality of gathers has a peak and two sloped sides, and one or both of the sides of one or more of the ridges bears at least one of the plurality of bonds.

5. The stretch laminate of any of the preceding examples wherein the one or more elastic members comprises a plurality of elastic strands disposed between the first web layer and the second web layer, with longitudinal axes being substantially parallel with the stretch direction and spaced apart in the trans-stretch direction by a strand spacing ES.

6. The stretch laminate of example 5 wherein the strand spacing is no greater than 14 mm, more preferably no greater than 10 mm, even more preferably no greater than 7 mm, and still more preferably no greater than 5 mm.

7. The stretch laminate of either of examples 5 or 6 wherein the length of the bonds is no greater than the strand spacing ES.

8. The stretch laminate of any of examples 5-7, wherein the bonds in the plurality of bonds are arranged in regularly-spaced columns, the columns having a column repeat interval CI, and wherein the column repeat interval CI is no greater than 1.5 ES, more no greater than 1.3 ES, 1.0 ES, 0.9 ES, and even more preferably no greater than 0.8 ES.

9. The stretch laminate of any of the preceding examples wherein the bonds in the plurality of bonds have a substantially straight rod shape.

10. The stretch laminate of any of the preceding examples wherein the nonwoven web material has a machine direction and a cross direction perpendicular to the machine direction, and the stretch direction is substantially parallel with the machine direction.

11. The stretch laminate of any of the preceding examples wherein the nonwoven web material has a machine direction and comprises spunlaid fibers.

12. The stretch laminate of example 11 wherein the spunlaid fibers have a machine direction bias.

13. The stretch laminate of example 12 wherein the machine direction is substantially parallel with the stretch direction.

14. The stretch laminate material of any of examples 5-8 wherein the elastic strands have been pre-strained by an amount from 50% to 200% prior to their incorporation into the stretch laminate material.

15. A disposable absorbent pant having an elasticized belt about a waist region, the belt comprising the stretch laminate material of any of the preceding examples.

The dimensions and values disclosed herein are not to be understood as being strictly limited to the exact numerical values recited. Instead, unless otherwise specified, each such dimension is intended to mean both the recited value and a functionally equivalent range surrounding that value.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A stretch laminate having a stretch direction and a trans-stretch direction perpendicular to the stretch direction, comprising:
a first web layer comprising a nonwoven web material bearing a pattern of thermal bonds comprising a plurality of bonds each having a length and a width, the length being oriented perpendicularly to the stretch direction and being greater than the width, for a majority of the bonds;
a second web layer; and
one or more elastic members disposed between the first web layer and the second web layer,
wherein the first web layer, the second web layer and the one or more elastic members together form the stretch laminate;
wherein the one or more elastic members have been joined with the first web layer and the second web layer while the one or more elastic members are pre-strained in the stretch direction; and
wherein, when the stretch laminate is in a relaxed condition, at least the first web layer comprises a plurality of gathers comprising elongate ridges and valleys oriented transversely to the stretch direction;
wherein the bonds in the pattern of thermal bonds on the nonwoven web material are distinct from any bonds or pattern of bonds used to bond the first and second layers of the stretch laminate together.

2. The stretch laminate of claim 1 wherein, for a majority of the bonds in the pattern, the length is greater than the width.

3. The stretch laminate of claim 2 wherein a majority of the bonds in the pattern have an aspect ratio of length to width of at least 2.

4. The stretch laminate of claim 1 wherein the pattern of bonds has a stretch direction repeat interval RDSD measured along an identifiable first line SDL parallel with the stretch direction, a trans-stretch direction repeat interval RDX measured along a second line XL perpendicular to the stretch direction, individual ones of the bonds arranged along the second line XL and within the trans-stretch direction repeat interval RDX have lengths, individual ones of the bonds arranged along the first line SDL and within the stretch direction repeat interval RDSD have widths, and the ratio of the sum of the lengths to the trans-stretch direction repeat interval RDX is greater than the ratio of the sum of the widths to the stretch direction repeat interval RDSD, for any identifiable second line SDL.

5. The stretch laminate of claim 1 wherein each ridge of the plurality of gathers has a peak and two sloped sides, and one or both of the sides of one or more of the ridges bears at least one of the plurality of bonds.

6. The stretch laminate of claim 1 wherein the one or more elastic members comprises a plurality of elastic strands disposed between the first web layer and the second web layer, with longitudinal axes being substantially parallel with the stretch direction and spaced apart in the trans-stretch direction by a strand spacing ES.

7. The stretch laminate of claim 6 wherein the strand spacing is no greater than 7 mm.

8. The stretch laminate of claim 6 wherein the length of the bonds is no greater than the strand spacing ES.

9. The stretch laminate of claim 6, wherein the bonds in the plurality of bonds are arranged in regularly-spaced columns, the columns having a column repeat interval CI, and wherein the column repeat interval CI is no greater than 1.5 ES, more no greater than 1.3 ES, 1.0 ES, 0.9 ES, and even more preferably no greater than 0.8 ES.

10. The stretch laminate of claim 1 wherein the bonds in the plurality of bonds have a substantially straight rod shape.

11. The stretch laminate of claim 1 wherein the nonwoven web material has a machine direction and a cross direction perpendicular to the machine direction, and the stretch direction is substantially parallel with the machine direction.

12. The stretch laminate of claim 1 wherein the nonwoven web material has a machine direction and comprises spunlaid fibers.

13. The stretch laminate of claim 12 wherein the spunlaid fibers have a machine direction bias.

14. The stretch laminate of claim 13 wherein the machine direction is substantially parallel with the stretch direction.

15. The stretch laminate material of claim 6 wherein the elastic strands have been pre-strained by an amount from 50% to 200% prior to their incorporation into the stretch laminate material.

16. A disposable absorbent pant having an elasticized belt about a waist region, the belt comprising the stretch laminate material of claim 1.
